(19) United States
(12) Patent Application Publication
(10) Pub. No.: US 2018/0306702 A1
(43) Pub. Date: Oct. 25, 2018

(54) DEVICE AND METHOD FOR MEASURING MECHANICAL PROPERTY OF CELL

(71) Applicant: Beijing Institute of Nanoenergy and Nanosystems, Beijing (CN)

(72) Inventors: Zhonglin WANG, Beijing (CN); Zhou LI, Beijing (CN); Qiang ZHENG, Beijing (CN); Junyi ZHAI, Beijing (CN); Mingceng PENG, Beijing (CN); Yalan ZHANG, Beijing (CN)

(21) Appl. No.: 15/771,889

(22) PCT Filed: Aug. 26, 2016
(86) PCT No.: PCT/CN2016/096929

§ 371 Date: Apr. 27, 2018

(30) Foreign Application Priority Data
Aug. 28, 2015 (CN) 201510542034.8

(51) Int. Cl. G01N 21/17; C12Q 1/02; G01N 33/483

(52) U.S. Cl. CPC G01N 21/17; B32B 5/00; G01N 33/483; C12Q 1/02

(57) ABSTRACT

Provided are a device and method for measuring mechanical properties of a cell, the device including: a substrate layer (2); and a nanowire layer (1) arranged on the substrate layer (2) and including a nanowire array, nanowires (11) in the array are configured to emit an light signal, and in response to a cell (3) to be tested being placed on the nanowire layer (1), a change in the light signal emitted by the nanowires (11) supporting the cell to be tested (3) represents mechanical properties of the corresponding cell. Mechanical properties of a cell can be measured in real time according to a change in the light signal emitted by the nanowires.

### CROSS-REFERENCE TO RELATED APPLICATIONS

This application is a Section 371 National Stage Application of International Application No. PCT/CN2016/096929, filed on 26 Aug. 2016 and entitled with “DEVICE AND METHOD FOR MEASURING MECHANICAL PROPERTY OF CELL”, and claims priority to Chinese Application No. 201510542034.8, filed on 28 Aug. 2015 and entitled with “DEVICE AND METHOD FOR MEASURING MECHANICAL PROPERTY OF CELL”, the contents of which are incorporated herein by reference in their entirety.

### TECHNICAL FIELD

The disclosure relates to a technical field of cell measurement, and in particular to a measurement device and a measuring method for measuring mechanical properties of a cell.

### DESCRIPTION OF THE RELATED ART

All biological tissues are composed of cells. The morphological structure and function of cells, the growth, development, maturation, increment, senescence, death, and carcinogenesis of cells, and the differentiation and regulatory mechanisms of cells are all related to the cell mechanical properties. When functions of cells are performed, the relevant genetic information may be used to synthesize, select, store and transport various biomolecules, to convert energy of various forms, to transmit signals of various forms, and to maintain or adjust their internal structures in response to effects of the external environment. All these above behaviors are related to the mechanical process. Therefore, it plays a very important role to understand and study cell biomechanics in the life science research at the cellular and molecular level.

Currently, cell mechanical properties are generally measured by means of a nanowire/micronwire array. For example, on basis of a PDMS (polydimethylsiloxane) micropillar array, cell mechanical properties are determined by measuring the amount of bending of the micropillar and the Young's modulus of the material. However, there are a lot of limitations in this method:

(1) The measuring method is constrained since the cells need to be fixed and observed by means of SEM (scanning electron microscope), which will not reflect the mechanical behaviors of living cells in real time;

(2) When the quantitative measurement of the nanowire deformation is carried out based on the photographs taken by the SEM, there are much interference due to the human factor and therefore the error was large; and

(3) The SEM is expensive and is not prone to a popularization.

### SUMMARY

The present disclosure provides a measurement device for measuring mechanical properties of a cell, including: a substrate layer; and a nanowire layer located on the substrate layer and including an array of nanowires, the nanowires in the array are configured to emit a light signal, and wherein in response to a cell to be tested being placed on the nanowire layer, the light signal emitted by the nanowires supporting the cell to be tested changes to characterize corresponding cell mechanical properties.

### DESCRIPTION OF THE DRAWINGS

The accompanying drawings are intended to provide a further understanding of the present disclosure, constitute a part of the specification, and together with the following detailed description, serve to explain the present disclosure, but the present disclosure will not be limited thereto. In the drawings:

FIG. 1 is a structural schematic view of a measurement device for measuring mechanical properties of a cell according to the present disclosure;.

FIG. 2 is a schematic view of change of a light signal of the measurement device for measuring mechanical properties of a cell according to the present disclosure at the time of measuring the cell to be tested;

FIG. 3 is a comparative diagram indicating the change of spectrum before and after the cell to be tested is applied; and

FIG. 4 is a dynamical front view of the measurement device for measuring mechanical properties of a cell according to the present disclosure.

### REFERENCE SIGNS:

1 nanowire layer;

11 nanowire;

2 substrate layer;

3 cell to be tested.

### DETAILED DESCRIPTION OF EMBODIMENTS

The specific embodiments of the present disclosure will be described in detail below with reference to the accompanying drawings. It should be understood that the specific embodiments described herein are only used to illustrate and explain the present disclosure and are not intended to limit the present disclosure.

The directional terms mentioned in the present disclosure, such as “upper”, “lower”, “front”, “rear”, “left”, “right”, and the like, are just the directions referring to the drawings. Therefore, the directional terms used are intended to be illustrative and not to limit the scope of the present disclosure.

The object of the present disclosure is to provide a measurement device and a measuring method for measuring mechanical properties of a cell, which can be used to determine cell mechanical properties in real time.

With the measurement device for measuring mechanical properties of a cell according to the present disclosure, since the nanowire layer capable of emitting light is provided, the cell to be tested can be directly placed on the nanowire layer, the cell mechanical signal can be converted into a visible light signal, and it will be convenient for the measurement. The mechanical properties of the cell to be tested can be precisely determined based on changed parameters of the light signal and thus a high accuracy is obtained.

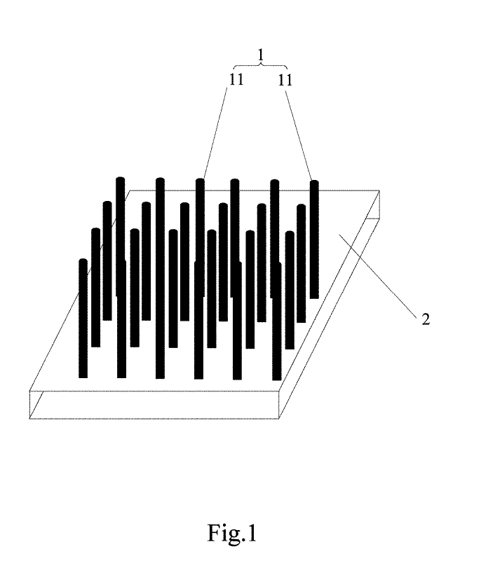

As shown in FIG. 1, the measurement device for measuring mechanical properties of a cell according to the present disclosure includes: a substrate layer 2; and a nanowire layer 1 located on the substrate layer 2 and including an array of nanowires, the nanowires in the nanowire array can emit a light signal, and after a cell 3 to be tested is placed on the nanowire layer 1 , the light signal emitted by the nanowires 11 supporting the cell 3 to be tested changes to characterize corresponding mechanical properties of the cell. The nanowire array of the nanowire layer 1 may be formed on the substrate layer 2 by liquid phase synthesis, vapor deposition, or etching.

With the measurement device for measuring mechanical properties of a cell according to the present disclosure, since the nanowire layer capable of emitting light is provided, the cell to be tested can be directly placed on the nanowire layer, the cell mechanical signal can be converted into a visible light signal, and it will be convenient for the measurement. The mechanical properties of the cell to be tested can be precisely determined based on changed parameters of the light signal and thus a high accuracy is obtained. In addition, in the measurement process, it is unnecessary to fix the cell and a real-time measurement of living cells can be achieved.

The changed parameters of the light signal include at least one of displacement amount of the light signal, intensity of the light signal, and spectrum variation of the light signal. The corresponding parameters are sensitive to the change, and the measurement accuracy is high, enabling a measurement of a tiny mechanical signal.

In the present disclosure, the shape of a single nanowire 11 does not affect the realization of the measurement function, so the nanowire 11 can be of any shape, such as a cone, a spindle, a cylinder, or a prism. In order to achieve an accurate measurement of single-cell-level mechanical properties, the length to diameter ratio or aspect ratio of the nanowires 11 ranges from 1:1 to 1:50; preferably, the length to diameter ratio or aspect ratio ranges from 1:3 to 1:10. Each nanowire 11 has a cross-sectional dimension of 50 nm to 1 μm; preferably, the cross-sectional dimension is 100 nm to 300 nm. When the nanowire is of a cylinder, the cross-sectional dimension is a diameter; when the nanowire is of a non-uniform structure such as a cone or a spindle, the cross-sectional dimension is the size of the thickest portion of the nanowire. Further, the distance between adjacent nanowires in the nanowire array is 50 nm to 50 μm; preferably, the distance between adjacent nanowires is 200 nm to 1 μm.

Each of the nanowires 11 in the nanowire layer 1 is made of a fluorescent material. The fluorescent material includes a fluorescent semiconductor material composed of a Group IIB-VIA element or a Group IIIA-VA element. For example, the semiconductor material may be: a two-component fluorescent semiconductor material such as ZnO, ZnS, ZnSe, GaN, InP, CdS, CdSe or the like, a multi-component fluorescent semiconductor material such as ZnCdSe, CdSeS or the like, and heterostructures formed from different semiconductor materials such as CdSe/ZnO, GaN/InP, but is not limited thereto.

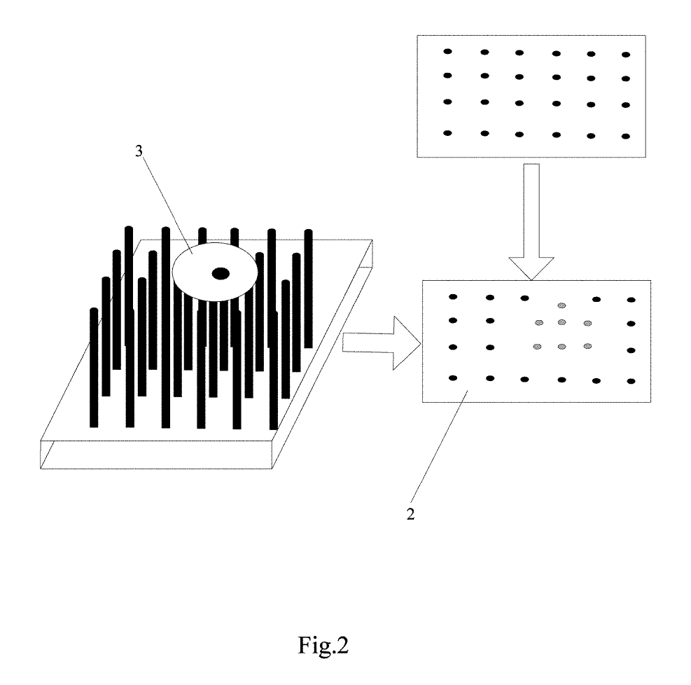

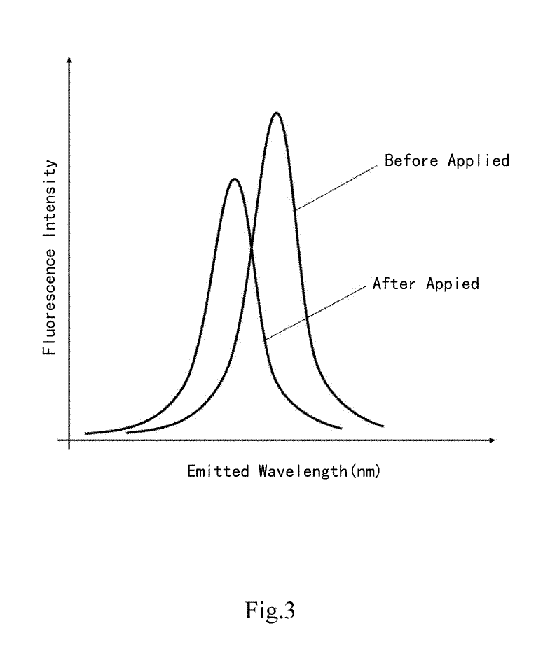

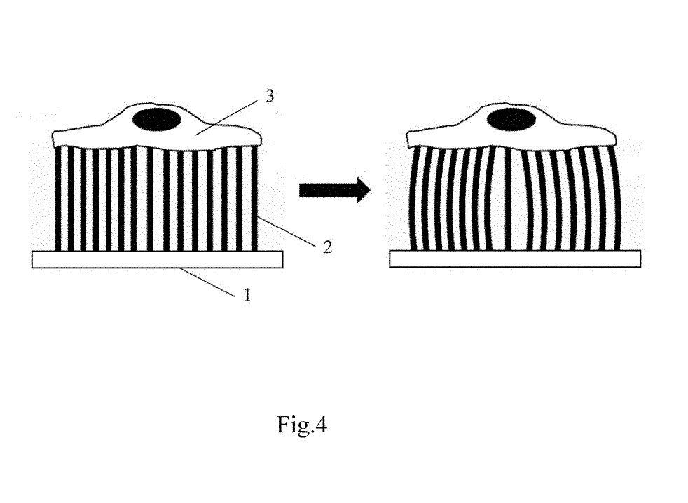

With the measurement device for measuring mechanical properties of a cell according to the present disclosure, after a light source having a corresponding waveband is stimulated, the nanowire can emit a corresponding light signal. Under the action of the cell mechanical behavior of the cell to be tested, the nanowire supporting the cell to be tested is changed. Based on the modulation effect of the piezo-phototronic, the intensity (as shown in FIG. 2) and the spectrum (as shown in FIG. 3) of the light signal change. When the mechanical behavior of the cell to be tested causes the corresponding nanowire to bend, the displacement of the corresponding light signal (as shown in FIGS. 2 and 4) changes. Thus, the magnitude and direction of the current cell force of the cell to be tested can be determined; and the cell mechanical properties can then be determined.

The cell mechanical properties include: proliferation (division), differentiation and migration of the cell induced by cytoskeleton and molecular motor; the movement and the change of shape during a cell signal transduction; and the electrostatic force and the Van der Waals force that are generated when a cell-cell interaction or a cell-environment interaction occurs. Depending on the purpose of the measurement, a specific stimulating factor may be applied directly to the cell to be tested or the cell culture environment so that the cell to be tested performs corresponding cell mechanical behavior(s).

In addition, depending on the difference in the compatibility, degradability, cell adhesion, and the like of the cell culture solution, the surface of the measurement device for measuring mechanical properties of a cell according to the present disclosure may be further processed to adapt to the cells and the environment in which they grows.

For example, the measurement device for measuring mechanical properties of a cell according to the present disclosure further includes a protective layer (not shown in the figures) disposed on a surface of each of the nanowires 11 in the nanowire layer 1 and coating the respective nanowires 11.The protective layer is a transparent or translucent thin layer, which is convenient for observing the change of the light signal. The protective layer generally has a thickness of less than 100 nm. The protective layer may be an inorganic plating layer made of an inorganic material such as aluminum oxide (AL₂O₃), which can effectively prevent the measurement device for measuring mechanical properties of a cell according to the present disclosure from being corroded and degraded in the cell culture liquid and prevent toxic ions from leaking, improving the stability and safety in use.

The method for preparing the inorganic plating layer may be performed by forming a transparent or translucent thin layer with a thickness less than 100 nm on the surface of the nanowires 11 through a common inorganic material plating method such as epitaxial growth, sputtering, atomic deposition, chemical deposition, vapor deposition or the like.

In addition, the protective layer may be an organic modified layer made of an organic material. For example, fibronectin can be used to increase the hydrophilicity of the device and the adhesion of the cell which is difficult to be adhered, such as primary cultured cardiomyocytes and nerve cells, to the nanowire layer, so that the culture state of the cells can be brought closer to a normal level.

In an embodiment, the method for preparing the organic modified layer may be performed by joining artificial or natural organic molecules to the surface of the nanowire to form an organic modified layer by means of assembly, adsorption, bonding, etc., so as to prevent corrosion and leakage of toxic ions and to increase the hydrophilicity and cell adhesion.

According to the present disclosure, depending on the preparation materials of the various components in the measurement device, the types of the cell, and different measurement environments, it may be chosen to coat the surface of the nanowire 11 with an inorganic plating layer or an organic modified layer and a corresponding material, which will not be particularly limited herein.

The disclosure also provides a method for measuring cell mechanical properties, including: placing a cell to be tested on the above-mentioned measurement device for measuring cell mechanical properties; obtaining a change of the light signal emitted by the nanowire layer in the measurement device for measuring cell mechanical properties to characterize the corresponding cell mechanical properties; determining a magnitude and a direction of a cell force of the cell to be tested based on the changed parameters of the light signal; and determining the cell mechanical properties of the current cell to be tested based on the magnitude and direction of the cell force.

Further, the method for measuring cell mechanical properties according to the present disclosure further includes sterilizing the measurement device for measuring cell mechanical properties before the cell to be tested is placed on the measurement device for measuring cell mechanical properties. An appropriate sterilization method such as high-pressure steam, irradiation, or drug treatment can be selected depending on the material properties of the measurement device for measuring cell mechanical properties.

In an embodiment, the step of placing a cell to be tested on the measurement device for measuring cell mechanical properties includes: placing the measurement device for measuring cell mechanical properties into a cell culture container (usually a culture dish) such that the cell to be tested is inoculated on a surface of the measurement device for measuring cell mechanical properties; and adherently growing the cell to be tested on the surface of the measurement device for measuring cell mechanical properties after the cell to be tested is cultured for a preset period of time.

The cell to be tested is joined to the contacted nanowires through adhesive molecules after it is adhered. When the cell performs a certain mechanical behavior, the deformation and movement of the cell membrane and the change of the internal skeleton stress will cause the corresponding nanowires to generate a strain. Depending on the purpose of measurement, a specific stimulating factor may be applied to the cell to be tested or the culture environment so that the cell to be tested performs corresponding mechanical behavior(s).

It is not necessary to use any expensive SEM in the measurement observation. It is sufficient to use an ordinary optical microscope (such as an inverted fluorescence microscope or a laser scanning confocal microscopy) which is convenient, efficient, of low cost, and widely used. For example, the measurement device with the cultured cell is placed below an inverted fluorescent microscope or a laser scanning confocal microscopy, and an appropriate range of laser irradiation is selected based on the optical characteristics of the nanowire material to perform a real-time observation. An array of light spots corresponding to period of the nanowire array is present in the microscope's field of view. The light signal emitted by the nanowires supporting the cell to be tested changes. The intensity of the light signal and the spectrum of the light signal are different from the light signal of the normally emitting nanowires therearound due to the piezo-phototronic effect. The reaction is sensitive. When the mechanical behavior of the cell to be tested is sufficient to cause the nanowires to bend, the corresponding position of the light signal is shifted. The cell mechanical properties can be observed and analyzed in real time based on the three variables of the displacement of the light signal, the change of the intensity of the light signal, and the spectral change of the light signal in combination with the physical properties of the material itself.

In the present disclosure, the response of the piezo-phototronic signal to the force is more sensitive than that of the conventional nanowire deformation parameters, which facilitates the detection of an even smaller change in the mechanical signal. The mechanical signal of the cell is converted into a visible light signal which can be observed under a microscope in a cell culture state, and the mechanical properties of living cells (such as beating of myocardial cells, migration of tumor cells, etc.) can be determined in real time by recording the continuously-changing light signal (position and intensity thereof). The analysis of cell mechanical properties achieved by measuring the displacement amount of the light signal and the change of the intensity of the light signal as well as the change of luminescence spectra is more scientific and accurate than the traditional single-variable analysis (the amount of deformation of nanowires). In addition, the changed parameters of the light signal can be directly obtained in a real-time observation, which also reduces the human error generated in the conventional method in which the measurement is indirectly performed through photographs.

The exemplary embodiments of the present disclosure have been described in detail above with reference to the accompanying drawings. However, the present disclosure is not limited to the specific details of the above embodiments. Various simple variations of the technical solutions according to the present disclosure can be made within the technical concept of the present disclosure. These simple variations all fall within the protection scope of the present disclosure.

In addition, it should be noted that the specific technical features described in the above specific embodiments can be combined in any suitable manner without contradiction. To avoid unnecessary repetition, various possibilities of combination will not be further described in the present disclosure.

In addition, any combination of various embodiments in the present disclosure may also be possible as long as it does not violate the idea of the present disclosure, and it should also be regarded as the disclosure of the present disclosure.

1. A measurement device for measuring cell mechanical properties, comprising:
a substrate layer; and
a nanowire layer located on the substrate layer and comprising an array of nanowires,
wherein the nanowires in the array are configured to emit a light signal; and
wherein in response to a cell to be tested being placed on the nanowire layer, the light signal emitted by the nanowires supporting the cell to be tested changes to characterize corresponding mechanical properties of the cell.

2. The measurement device for measuring cell mechanical properties according to claim 1,
wherein changed parameters of the light signal comprise at least one of displacement amount of the light signal, intensity of the light signal, and spectrum variation of the light signal.

3. The measurement device for measuring cell mechanical properties according to claim 1, wherein the measurement device further comprises a protective layer disposed on a surface of each of the nanowires in the nanowire layer and coating the respective nanowires.

4. The measurement device for measuring cell mechanical properties according to claim 3,
wherein the protective layer is in a transparent or translucent state.

5. The measurement device for measuring cell mechanical properties according to claim 3,
wherein the protective layer has a thickness of less than 100 nm.

6. The measurement device for measuring cell mechanical properties according to claim 3,
wherein the protective layer is an inorganic plating layer or an organic modified layer.

7. The measurement device for measuring cell mechanical properties according to claim 1, wherein each of the nanowires in the nanowire layer is made of a fluorescent material.

8. The measurement device for measuring cell mechanical properties according to claim 7,
wherein the fluorescent material includes a fluorescent semiconductor material composed of a Group IIB-VIA element or a Group IIIA-VA element.

9. The measurement device for measuring cell mechanical properties according to claim 1,
wherein the aspect ratio of each of the nanowires ranges from 1:1 to 1:50.

10. The measurement device for measuring cell mechanical properties according to claim 9,
wherein the aspect ratio of each of the nanowires ranges from 1:3 to 1:10.

11. The measurement device for measuring cell mechanical properties according to claim 1,
wherein each nanowire has a cross-sectional dimension of 50 nm to 1 μm.

12. The measurement device for measuring cell mechanical properties according to claim 11,
wherein each nanowire has a cross-sectional dimension of 100 nm to 300 nm.

13. The measurement device for measuring cell mechanical properties according to claim 1,
wherein a distance between adjacent nanowires is 50 nm to 50 μm.

14. The measurement device for measuring cell mechanical properties according to claim 13,
wherein the distance between adjacent nanowires is 200 nm to 1 μm.

15. A method for measuring cell mechanical properties, comprising:
placing a cell to be tested on the measurement device for measuring cell mechanical properties according to claim 1; and
obtaining a change of the light signal emitted by the nanowire layer in the measurement device to characterize the corresponding mechanical properties of the cell.

16. The method for measuring cell mechanical properties according to claim 15,
wherein the method further comprises:
determining a magnitude and a direction of a cell force of the cell to be tested based on changed parameters of the light signal; and
determining the cell mechanical properties of the current cell to be tested based on the magnitude and direction of the cell force.

17. The method for measuring cell mechanical properties according to claim 15,
wherein the measuring method further comprises:
sterilizing the measurement device for measuring cell mechanical properties before the cell to be tested is placed on the measurement device for measuring cell mechanical properties.

18. The method for measuring cell mechanical properties according to claim 15,
wherein placing a cell to be tested on the measurement device for measuring cell mechanical properties comprises:
placing the measurement device for measuring cell mechanical properties into a cell culture container such that the cell to be tested is inoculated on a surface of the measurement device for measuring cell mechanical properties; and
adherently growing the cell to be tested on the surface of the measurement device for measuring cell mechanical properties after the cell to be tested is cultured for a preset period of time.

19. The method for measuring cell mechanical properties according to claim 15, wherein the method further comprises:
applying a stimulating factor to the cell to be tested or a culture environment so that the cell to be tested performs a corresponding cell mechanical behavior.

20. The measurement device for measuring cell mechanical properties according to claim 2,
wherein the measurement device further comprises a protective layer disposed on a surface of each of the nanowires in the nanowire layer and coating the respective nanowires.
